(19) United States
(12) Patent Application Publication
(10) Pub. No.: US 2026/0274863 A1
(43) Pub. Date: Sep. 17, 2026

(54) Photochromic Compound and Its Preparation Method and Application

(71) Applicant: SHANGHAI GANTIAN OPTICAL MATERIAL CO., LTD, Shanghai (CN)

(72) Inventor: Jiaan GAN, Shanghai (CN)

(21) Appl. No.: 19/674,153

(22) Filed: May 12, 2026

Related U.S. Application Data
(63) Continuation of application No. PCT/CN2025/101941, filed on Jun. 19, 2025.

(30) Foreign Application Priority Data
Jan. 7, 2025 (CN) 202510018919.1

(51) Int. Cl. C07D 513/04; C07D 491/044; C07D 498/04

(52) U.S. Cl. CPC C07D 513/04; C07D 491/044; C07D 498/04

(57) ABSTRACT

The present application relates to the technical field of photochromic materials, and provides a photochromic compound, as well as a preparation method and application thereof. R₁, R₂, R₇ and R₈ of the structural formula of the photochromic compound of the present application are each independently selected from hydrogen, halogen, hydroxyl, alkyl, alkoxy, amino, aryl, or heteroaryl; R₃ and R₄ are each independently selected from hydrogen, halogen, hydroxyl, alkyl, heterocycloalkyl, alkoxy, aryl, or heteroaryl; R is selected from hydrogen, alkyl, cycloalkyl, or aryl; X is N—R₉, O, or S; and R₉ is selected from hydrogen, alkyl, aryl, or heteroaryl. The photochromic compound of the present application can exhibit a color-change effect under light irradiation, and can return to a colorless and transparent state indoors or in the dark.

### CROSS-REFERENCE TO RELATED APPLICATIONS

This application is a continuation of International Application No. PCT/CN2025/101941, filed on Jun. 19, 2025, which claims priority to Chinese Patent Application No. CN202510018919.1, filed on Jan. 7, 2025, the contents of which are incorporated herein by reference in their entirety.

### TECHNICAL FIELD

The present application relates to the technical field of photochromic materials, and in particular to a photochromic compound and its preparation method and application.

### BACKGROUND

With the improvement of building energy efficiency standards and the increasing demand for comfort in living and working environments, infrared absorption functionality has gradually expanded to multiple fields including architectural glass, automotive glass, electronic device protective glass, and others. Glass itself is a transparent material, and traditional glass has limited infrared absorption capability, especially for near-infrared rays. During the glass production and manufacturing process, specific absorbing materials are typically added to enhance the glass's ability to absorb infrared rays. The absorbing materials are uniformly dispersed into the glass matrix, enabling the glass to effectively absorb infrared rays within specific wavelength ranges, thereby achieving the purpose of thermal insulation or reducing infrared penetration. Commonly used absorbing materials include metal oxides (such as iron oxide, cobalt oxide, etc.) and rare earth element compounds.

Under intense light or strong ultraviolet irradiation, a photochromic material undergoes a structural change, causing the glass to darken and reducing the transmittance of visible light and infrared radiation. When the light intensity decreases, the photochromic material returns to its initial structure, and the glass becomes transparent again. The self-adaptive regulation function of photochromic materials in response to light intensity can effectively alleviate indoor glare and improve visual comfort.

Therefore, developing photochromic materials with a broad absorption range and applying them to glass or interlayers to enhance user comfort has broad application prospects.

### SUMMARY

The objective of the present application is to provide a photochromic compound and its preparation method and application in order to address the deficiencies of the prior art.

To achieve the above objective, the present application provides the following technical solutions:

The present application provides a photochromic compound, wherein the structural formula of the photochromic compound is as follows:

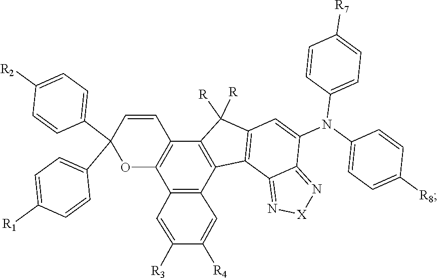

wherein R₁, R₂, R₇, and R₅ are each independently selected from hydrogen, halogen, hydroxyl, alkyl, alkoxy, amino, aryl, or heteroaryl;
wherein R₃ and R₄ are each independently selected from hydrogen, halogen, hydroxyl, alkyl, heterocycloalkyl, alkoxy, aryl, or heteroaryl;
wherein R is selected from hydrogen, alkyl, cycloalkyl, or aryl;
wherein X is N—R₉, O, or S;
wherein R₉ is selected from hydrogen, alkyl, aryl, or heteroaryl.

Preferably, the photochromic compound is any one of the compounds represented by the following structural formulas:

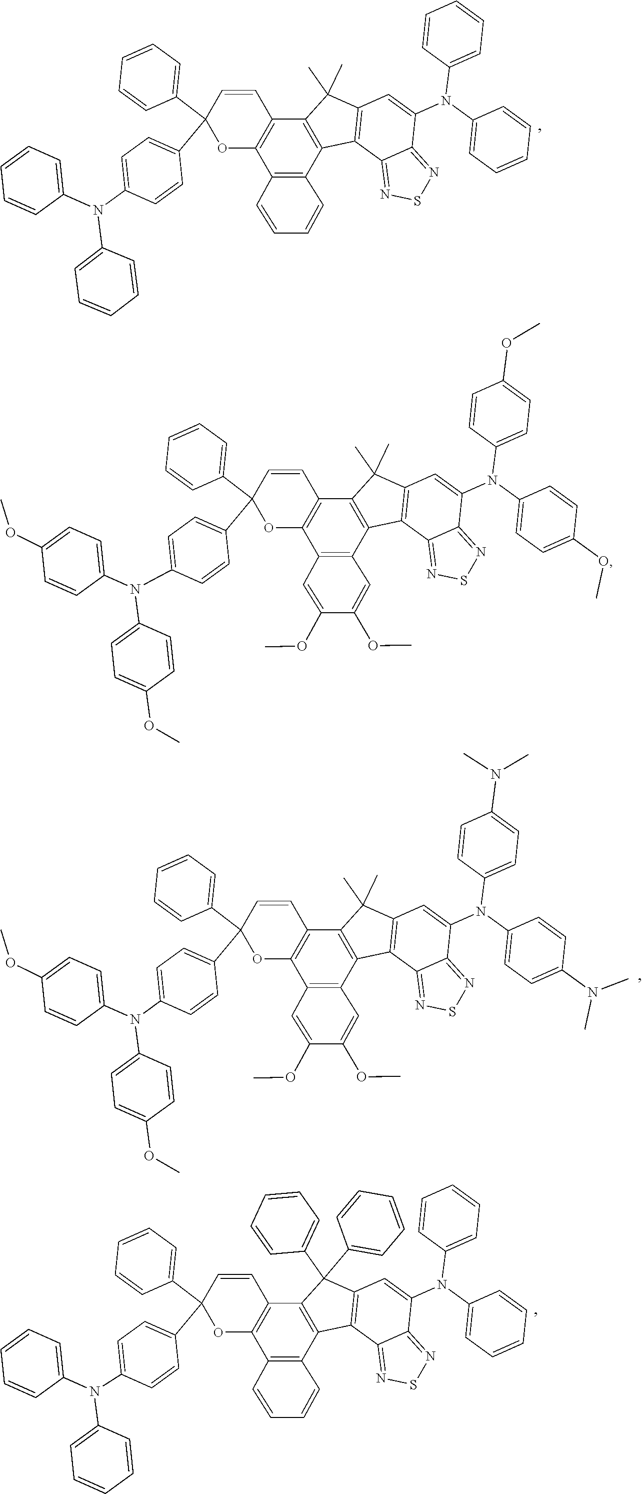

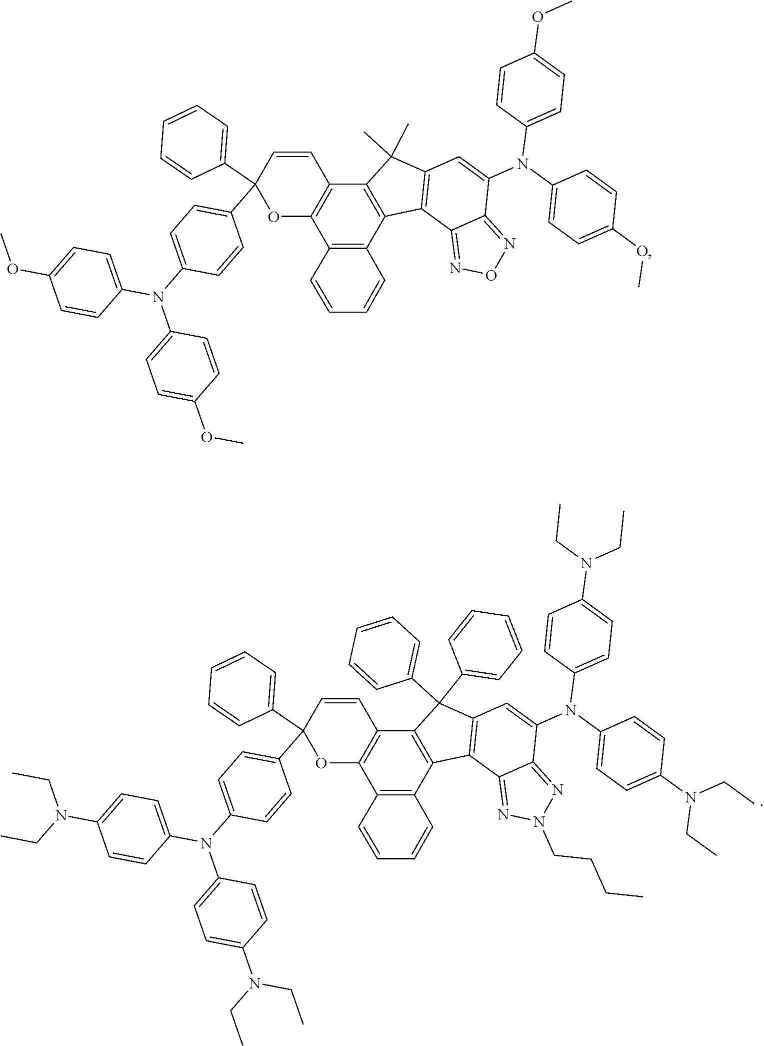 [SMILES: COc1ccc(N(c2ccc(OC)cc2)c2ccc(C3(c4ccccc4)C=Cc4c5c(c6ccccc6c4O3)-c3c(cc(N(c4ccc(OC)cc4)c4ccc(OC)cc4)c4nonc34)C5(C)C)cc2)cc1 ; CCCCn1nc2c(N(c3ccc(N(CC)CC)cc3)c3ccc(N(CC)CC)cc3)cc3c(c2n1)-c1c(c2c(c4ccccc14)OC(c1ccccc1)(c1ccc(N(c4ccc(N(CC)CC)cc4)c4ccc(N(CC)CC)cc4)cc1)C=C2)C3(c1ccccc1)c1ccccc1]

The present application further provides a method for preparing the photochromic compound, comprising the following steps:
(1) subjecting compound A. [1,1,-bis(diphenylphosphino) ferrocene]palladium (II) dichloride (Pd(dppf)Cl₂), bis(pinacolato)diboron, potassium acetate, toluene, and water are subjected to a Miyaura borylation reaction to afford an intermediate product;
(2) subjecting the intermediate product, compound B, tetrakis (triphenylphosphine)palladium (0) (Pd(PPh3)₄), an aqueous potassium carbonate solution, and tetrahydrofuran are subjected to a Suzuki coupling reaction to afford compound C;
(3) mixing compound C, a Grignard reagent, and tetrahydrofuran are mixed and allowed to react to afford compound D;
(4) subjecting compound D, pyridinium p-toluenesulfonate (PPTS), and chloroform are subjected to a cyclization reaction to afford compound E;
(5) subjecting compound E, compound F, tris(dibenzylideneacetone)dipalladium (0) (Pd₂(dba)₃), cesium carbonate, toluene, and water are subjected to a cross-coupling reaction to afford compound G;
(6) subjecting compound G, boron tribromide (BBr₃), and chloroform are subjected to a demethylation reaction to afford compound H;
(7) mixing compound H, compound J, pyridinium p-toluenesulfonate (PPTS), and dichloromethane are mixed and allowed to react, thereby obtaining the photochromic compound.

Preferably, the structural formula of compound A described in step (1) is as follows:

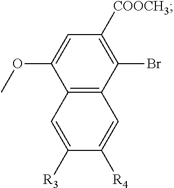

wherein R₃ and R₄ are each independently selected from hydrogen, halogen, hydroxyl, alkyl, heterocycloalkyl, alkoxy, aryl, or heteroaryl.

In step (1), molar the ratio of compound A, [1,1′-bis(diphenylphosphino)ferrocene]palladium (II) dichloride and bis(pinacolato)diboron is 48-52:1:58-63; and the mass ratio of compound A, potassium acetate, toluene, and water is 48-52 mmol: 1-2 g: 120-180 g: 10-15 g. The temperature of the Miyaura borylation reaction is 70-90° C., and the reaction time is 20-28 h.

Preferably, the structural formula of compound B described in step (2) is as follows:

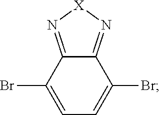

wherein X is N—R₉, O, or S, and R₉ is selected from hydrogen, alkyl, aryl, or heteroaryl.

In step (2), the molar ratio of compound B, tetrakis(triphenylphosphine)palladium (0) (Pd(PPh3)₄), and compound A described in step (1) is 75-80:2:48-52; and the mass ratio of the potassium carbonate solution, tetrahydrofuran, and compound A described in step (1) is 48-52 g: 200-300 g: 48-52 mmol. The concentration of the potassium carbonate solution is 1.5-2.5 mol/L. The temperature of the Suzuki coupling reaction is 50-70° C., and the reaction time is 12-24 h.

Preferably, the Grignard reagent described in step (3) has the structural formula R—Mg—Br, wherein R is selected from hydrogen, alkyl, cycloalkyl, or aryl.

In step (3), the molar/volume ratio of compound C, the Grignard reagent, and tetrahydrofuran is 32-38 mmol: 72-78 mmol: 250 mL. The reaction temperature is 20-30° C., and the reaction time is 14-24 h.

In step (4), the molar ratio of pyridinium p-toluenesulfonate to compound C described in step (3) is 12-16:32-38; and the molar/volume ratio of pyridinium p-toluenesulfonate to chloroform is 12-16 mmol: 100 mL. The temperature of the cyclization reaction is 20-30° C., and the reaction time is 1-3 h.

Preferably compound F described in step (5) is as follows:

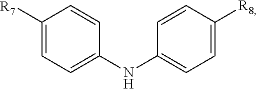

wherein R and R are each independently selected from hydrogen, halogen, hydroxyl, alkyl, alkoxy, amino, aryl, or heteroaryl. In step (5), the molar amounts of compound E, compound F, tris(dibenzylideneacetone) dipalladium (0), cesium carbonate, toluene, and water are in a ratio of 15-20 mmol: 25-30 mmol: 0.5-1.5 mmol: 34-38 mmol: 250 g: 20-30 g. The temperature of the cross-coupling reaction is 90-110° C., and the reaction time is 16-24 h.

Preferably, in step (6), the molar/volume ratio of compound G, boron tribromide, and chloroform is 10-15 mmol: 15-20 mmol: 10-25 mL. The temperature of the demethylation reaction is 20-30° C., and the reaction time is 16-24 h.

Preferably, the structural formula of compound J described in step (7) is as follows:

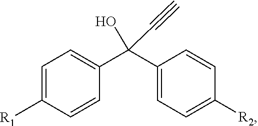

wherein R₁ and R₂ are each independently selected from the group consisting of hydrogen, halogen, hydroxyl, alkyl, alkoxy, amino, aryl, and heteroaryl.

In step (7), the molar/volume ratio of compound H, compound J, pyridinium p-toluenesulfonate, and dichloromethane is 2-5 mmol: 3-6 mmol: 1-4 mmol: 100 mL. The reaction temperature is 35-45° C., and the reaction time is 4-10 h.

The present application further provides an application of the photochromic compound in photochromic products.

The beneficial effects of the present application include, but are not limited to, the following aspects:
(1) The photochromic compound of the present application can exhibit a color-change effect under light irradiation, and can return to a colorless and transparent state indoors or in the dark.
(2) By introducing an electron-withdrawing benzo-fused heterocyclic structure into the parent framework and extending the conjugated structure, the photochromic compound of the present application can induce an intramolecular charge-transfer effect even in the near-infrared region, thereby achieving the color-change effect while effectively reducing heat exchange.

### DETAILED DESCRIPTION OF THE EMBODIMENTS

The present application provides a photochromic compound, wherein the structural formula of the photochromic compound is as follows:

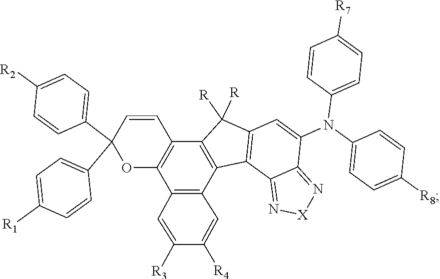

wherein R₁, R₂, R₇, and R₈ are each independently selected from hydrogen, halogen, hydroxyl, alkyl, alkoxy, amino, aryl, or heteroaryl;
R₃ and R₄ are each independently selected from hydrogen, halogen, hydroxyl, alkyl, heterocycloalkyl, alkoxy, aryl, or heteroaryl;
R is selected from hydrogen, alkyl, cycloalkyl, or aryl;
X is N—R₉, O, or S;
R₉ is selected from hydrogen, alkyl, aryl, or heteroaryl.

In the present application, the photochromic compound is preferably any one of the compounds represented by the following structural formulas:

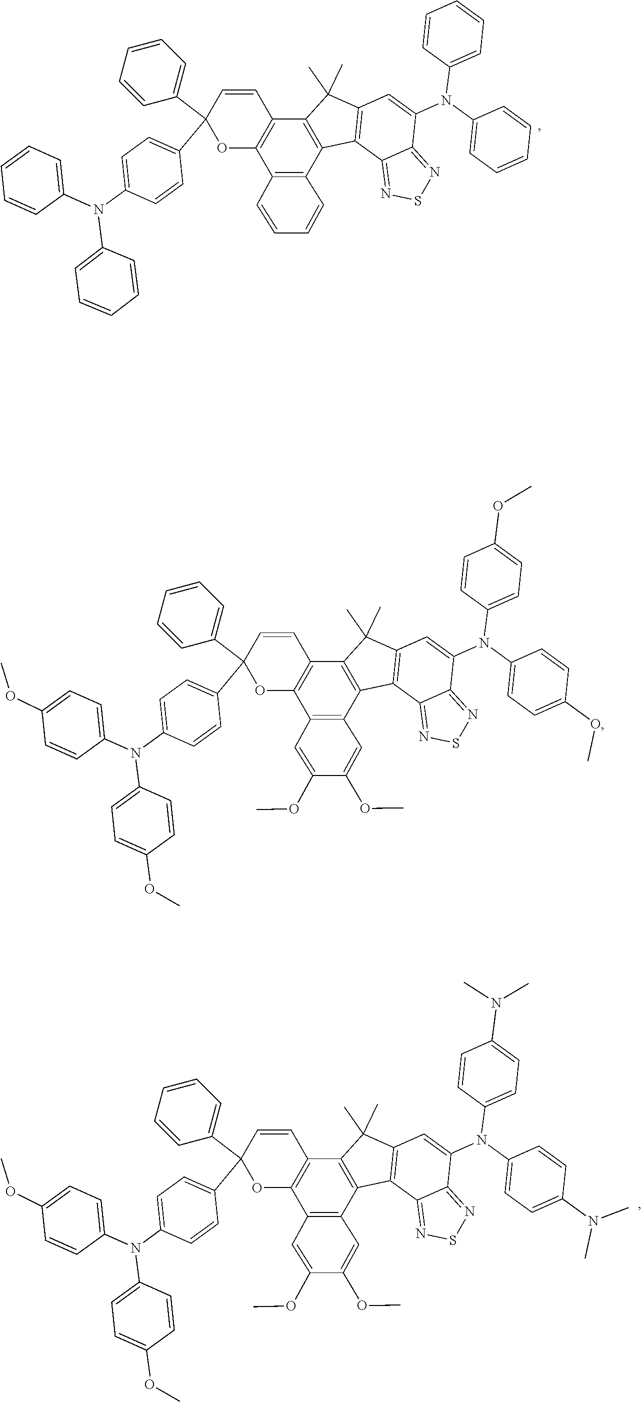

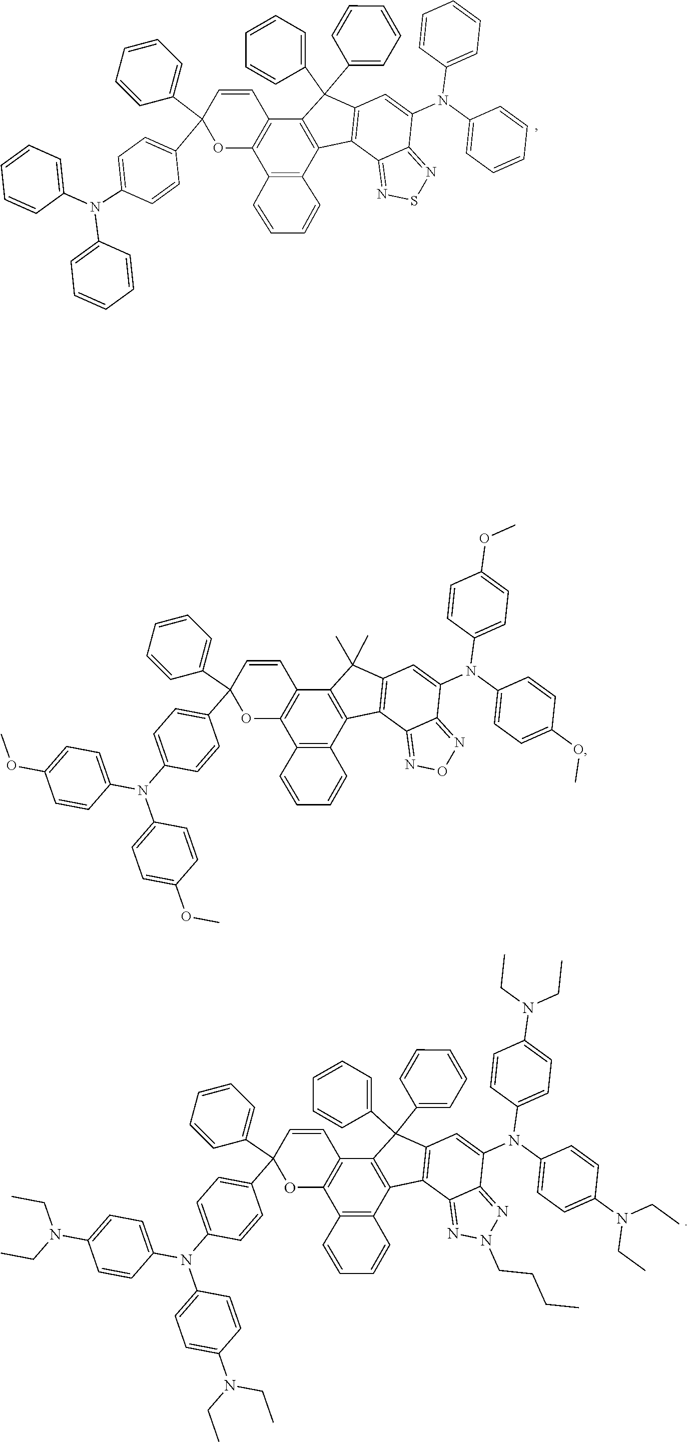 [SMILES: C1=CC(c2ccccc2)(c2ccc(N(c3ccccc3)c3ccccc3)cc2)Oc2c1c1c(c3ccccc23)-c2c(cc(N(c3ccccc3)c3ccccc3)c3nsnc23)C1(c1ccccc1)c1ccccc1 ; COc1ccc(N(c2ccc(OC)cc2)c2ccc(C3(c4ccccc4)C=Cc4c5c(c6ccccc6c4O3)-c3c(cc(N(c4ccc(OC)cc4)c4ccc(OC)cc4)c4nonc34)C5(C)C)cc2)cc1 ; CCCCn1nc2c(N(c3ccc(N(CC)CC)cc3)c3ccc(N(CC)CC)cc3)cc3c(c2n1)-c1c(c2c(c4ccccc14)OC(c1ccccc1)(c1ccc(N(c4ccc(N(CC)CC)cc4)c4ccc(N(CC)CC)cc4)cc1)C=C2)C3(c1ccccc1)c1ccccc1]

The present application further provides a method for preparing the photochromic compound, comprising the following steps:
(1) subjecting compound A, [1,1,-bis(diphenylphosphino) ferrocene]palladium (II) dichloride (Pd(dppf)Cl₂), bis(pinacolato)diboron, potassium acetate, toluene, and water are subjected to a Miyaura borylation reaction to afford an intermediate product;
(2) subjecting the intermediate product, compound B, tetrakis (triphenylphosphine)palladium (0) (Pd(PPh3)₄), an aqueous potassium carbonate solution, and tetrahydrofuran are subjected to a Suzuki coupling reaction to afford compound C;
(3) mixing compound C, a Grignard reagent, and tetrahydrofuran are mixed and allowed to react to afford compound D;
(4) subjecting compound D, pyridinium p-toluenesulfonate (PPTS), and chloroform are subjected to a cyclization reaction to afford compound E;
(5) subjecting compound E, compound F, tris(dibenzylideneacetone)dipalladium (0) (Pd₂(dba)₃), cesium carbonate, toluene, and water are subjected to a cross-coupling reaction to afford compound G;
(6) subjecting compound G, boron tribromide (BBr₃), and chloroform are subjected to a demethylation reaction to afford compound H;
(7) mixing compound H, compound J, pyridinium p-toluenesulfonate, and dichloromethane are mixed and allowed to react, thereby obtaining the photochromic compound.

In the present application, the preferred synthetic route for the photochromic compound is as follows:

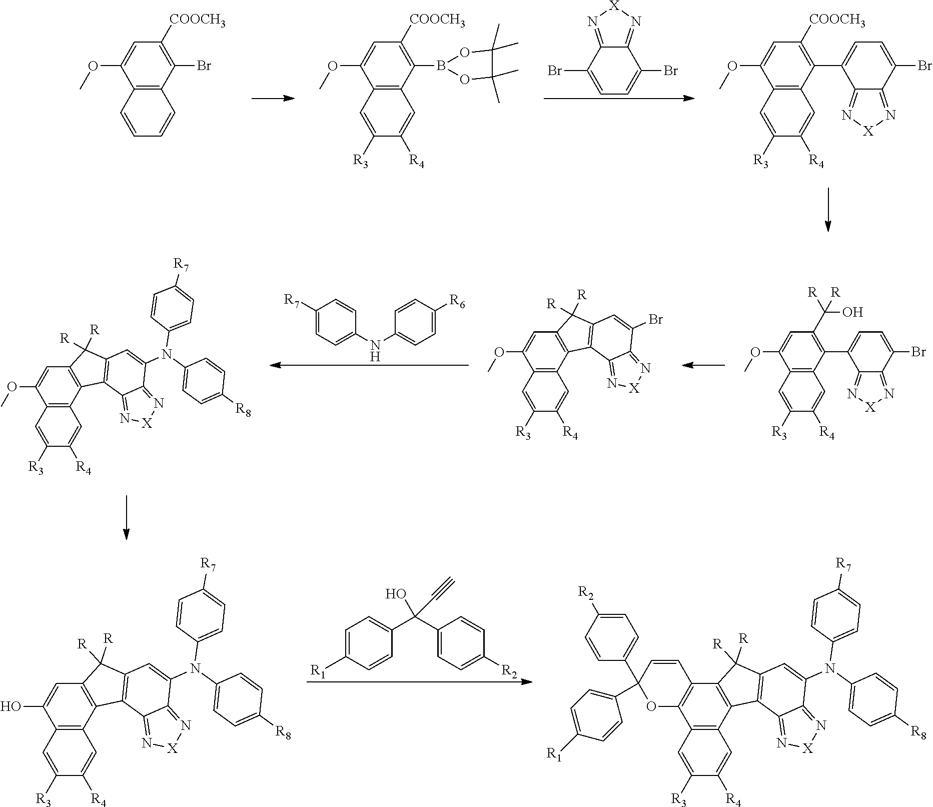

In the present application, the structural formula of compound A described in step (1) is as follows:

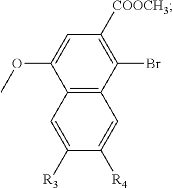

wherein R₃ and R₄ are each independently preferably selected from hydrogen, halogen, hydroxyl, alkyl, heterocycloalkyl, alkoxy, aryl, or heteroaryl.

In step (1), the amounts of compound A compound A, [1,1′-bis(diphenylphosphino)ferrocene]palladium (II) dichloride, and bis(pinacolato)diboron are such that the corresponding molar ratio is preferably 48-52:1:58-63, further preferably 49-51:1:59-62, and more preferably 50.8:1:61. The amounts of compound A, potassium acetate, toluene, and water are preferably 48-52 mmol, 1-2 g, 120-180 g, and 10-15 g, respectively; further preferably 49-51 mmol, 1.25-1.75 g, 135-165 g, and 11-14 g, respectively; and more preferably 50.8 mmol, 1.5 g, 150 g, and 12-13.5 g, respectively. The temperature of the Miyaura borylation reaction is preferably 70-90° C., further preferably 75-85° C., and more preferably 80° C. The reaction time of the Miyaura borylation reaction is preferably 20-28 h, further preferably 22-26 h, and more preferably 24 h.

In the present application, the Miyaura borylation reaction described in step (1) is preferably carried out under a nitrogen atmosphere, and stirring is preferably performed during the reaction. The stirring speed is preferably 150-250 rpm, and further preferably 200 rpm.

In the present application, after completion of the Miyaura borylation reaction described in step (1), the reaction mixture is preferably first cooled to room temperature and then subjected to rotary evaporation for concentration. The vacuum degree during rotary evaporation is preferably −0.05 to −0.01 MPa, and further preferably −0.03 to −0.02 MPa. The temperature for rotary evaporation is preferably 45-55° C., and further preferably 50° C. The rotary evaporation time is preferably 20-40 min, and further preferably 30 min.

In the present application, the structural formula of the intermediate product described in step (1) is as follows:

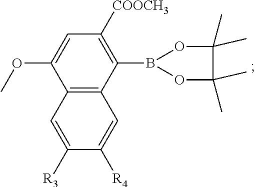

wherein R₃ and R₄ are each independently preferably selected from hydrogen, halogen, hydroxyl, alkyl, heterocycloalkyl, alkoxy, aryl, or heteroaryl.

In the present application, the structural formula of compound B described in step (2) is as follows:

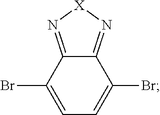

wherein X is preferably N—R₉, O, or S, and R₉ is selected from hydrogen, alkyl, aryl, or heteroaryl.

In step (2), the molar ratio of compound B, tetrakis(triphenylphosphine)palladium (0), and compound A described in step (1) is preferably 75-80:2:48-52, further preferably 76.2-79:2:49-51, and more preferably 77-78:2:50.8. The amounts of the potassium carbonate solution, tetrahydrofuran, and compound A described in step (1) are preferably 48-52 g, 200-300 g, and 48-52 mmol, respectively; further preferably 49-51 g, 220-280 g, and 49-51 mmol, respectively; and more preferably 50 g, 250 g, and 50.8 mmol, respectively. The concentration of the potassium carbonate solution is preferably 1.5-2.5 mol/L, further preferably 1.75-2.25 mol/L, and more preferably 2 mol/L. The temperature of the Suzuki coupling reaction is preferably 50-70° C., further preferably 55-65° C., and more preferably 60° C. The reaction time of the Suzuki coupling reaction is preferably 12-24 h, further preferably 16-20 h, and more preferably 18 h.

In the present application, the Suzuki coupling reaction described in step (2) is preferably carried out under a nitrogen atmosphere, and stirring is preferably performed during the reaction. The stirring speed is preferably 150-250 rpm, and further preferably 200 rpm.

In the present application, after completion of the Suzuki coupling reaction described in step (2), a mixed solution is obtained. Preferably, the mixed solution is first poured into hydrochloric acid, followed by sequential dilution, washing, drying, rotary evaporation for concentration, and silica gel column chromatography. The volume/molar ratio of hydrochloric acid to compound A described in step (1) is preferably 180-220 mL: 48-52 mmol, further preferably 190-210 mL: 49-51 mmol, and more preferably 200 mL: 50.8 mmol. The concentration of hydrochloric acid is preferably 1.5-2.5 mol/L, and further preferably 2 mol/L. Dilution is preferably carried out using ethanol, and the volume/molar ratio of ethanol to compound A is preferably 480-520 mL: 48-52 mmol, further preferably 490-510 mL: 49-51 mmol, and more preferably 500 mL: 50.8 mmol. Washing is preferably performed on the organic phase obtained after dilution, and the washing agents are preferably used sequentially as water and a sodium chloride solution, wherein the mass concentration of the sodium chloride solution is preferably 10-27%, and further preferably 26.5%. Drying is preferably carried out using anhydrous sodium sulfate. The vacuum degree during rotary evaporation is preferably −0. 05 to −0.01 MPa, and further preferably −0.03 to −0.02 MPa. The temperature for rotary evaporation is preferably 30-60° C., and further preferably 40° C. The eluent used for silica gel column chromatography is preferably n-hexane and dichloromethane, and the volume ratio of n-hexane to dichloromethane is preferably 6-9:3, and further preferably 7-8:3.

In the present application, the structural formula of compound C described in step (2) is as follows:

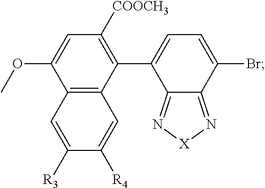

wherein R₃ and R₄ are each independently preferably selected from hydrogen, halogen, hydroxyl, alkyl, heterocycloalkyl, alkoxy, aryl, or heteroaryl; X is preferably N—R₉, O, or S; and R₉ is preferably selected from hydrogen, alkyl, aryl, or heteroaryl.

In the present application, the Grignard reagent described in step (3) has the structural formula R—Mg—Br, wherein R is preferably selected from hydrogen, alkyl, cycloalkyl, or aryl.

In step (3), the molar/volume ratio of compound C, the Grignard reagent, and tetrahydrofuran is preferably 32-38 mmol: 72-78 mmol: 250 mL, further preferably 33-37 mmol: 73-77 mmol: 250 mL, and more preferably 34.9-36 mmol: 75 mmol: 250 mL. The reaction temperature is preferably 20-30° C., further preferably 22-28° C., and more preferably 25° C. The reaction time is preferably 14-24 h, further preferably 16-20 h, and more preferably 19 h.

In step (4), the molar ratio of pyridinium p-toluenesulfonate to compound C described in step (3) is preferably 12-16:32-38, further preferably 13-15:33-37, and more preferably 14:34.9-36. The molar/volume ratio of pyridinium p-toluenesulfonate to chloroform is preferably 12-16 mmol: 100 mL, further preferably 13-15 mmol: 100 mL, and more preferably 14 mmol: 100 mL. The temperature of the cyclization reaction is preferably 20-30° C., further preferably 22-28° C., and more preferably 25° C. The reaction time of the cyclization reaction is preferably 1-3 h, further preferably 1.5-2.5 h, and more preferably 2 h.

In the present application, the reaction described in step (3) is preferably carried out under a nitrogen atmosphere, and stirring is preferably performed during the reaction. The stirring speed is preferably 150-250 rpm, and further preferably 200 rpm.

In the present application, in step (3), compound C and tetrahydrofuran are preferably first mixed, followed by dropwise addition of a solution of the Grignard reagent. The dropwise addition time is preferably 20-40 min, and further preferably 30 min. The solvent used for the Grignard reagent solution is preferably ethanol, and the concentration of the Grignard reagent in the solution is preferably 0.8-1.2 mol/L, and further preferably 1 mol/L.

In the present application, after completion of the reaction described in step (3), a mixed solution is obtained. Preferably, the mixed solution is first poured into hydrochloric acid, followed by sequential dilution, washing, drying, rotary evaporation for concentration, and silica gel column chromatography. The volume/molar ratio of hydrochloric acid to compound C is preferably 180-220 mL: 32-38 mmol, further preferably 190-210 mL: 33-37 mmol, an d more preferably 200 mL: 34.9-36 mmol. The concentration of hydrochloric acid is preferably 1.5-2.5 mol/L, and further preferably 2 mol/L. Dilution is preferably carried out using ethanol, and the volume/molar ratio of ethanol to compound C is preferably 480-520 mL: 32-38 mmol, further preferably 490-510 mL: 33-37 mmol, and more preferably 500 mL: 34.9-36 mmol. Washing is preferably performed on the organic phase obtained after dilution, and the washing agents are preferably used sequentially as water and a sodium chloride solution, wherein the mass concentration of the sodium chloride solution is preferably 10-27%, and further preferably 26.5%. Drying is preferably carried out using anhydrous sodium sulfate. The vacuum degree during rotary evaporation is preferably −0.05 to −0.01 MPa, and further preferably −0.03 to −0.02 MPa. The temperature for rotary evaporation is preferably 30-60° C., and further preferably 40° C. The rotary evaporation time is preferably 20-40 min, and further preferably 30 min. The eluent used for silica gel column chromatography is preferably n-hexane and dichloromethane, and the volume ratio of n-hexane to dichloromethane is preferably 5-7:4, and further preferably 6:4.

In the present application, the structural formula of compound D described in step (3) is as follows:

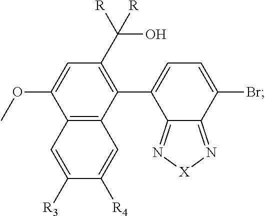

wherein R₃ and R₄ are each independently preferably selected from hydrogen, halogen, hydroxyl, alkyl, heterocycloalkyl, alkoxy, aryl, or heteroaryl; R is preferably selected from hydrogen, alkyl, cycloalkyl, or aryl; X is preferably N—R₉, O, or S; and R₉ is preferably selected from hydrogen, alkyl, aryl, or heteroaryl.

In the present application, during the cyclization reaction described in step (4), stirring is preferably performed. The stirring speed is preferably 150-250 rpm, and further preferably 200 rpm.

In the present application, after completion of the cyclization reaction described in step (4), a mixed solution is obtained. Preferably, the mixed solution is poured into water, and the mixture is stirred to obtain an organic phase. The organic phase is then sequentially subjected to washing, drying, rotary evaporation for concentration, and silica gel column chromatography. The volume/molar ratio of water to compound C described in step (3) is preferably 200-1000 mL: 32-38 mmol, further preferably 400-600 mL: 33-37 mmol, and more preferably 500 mL: 34.9-36 mmol. The stirring speed is preferably 100-400 rpm, and further preferably 200 rpm. The stirring time is preferably 5-15 min, and further preferably 10 min. The washing agent is preferably a sodium chloride solution, and the mass concentration of the sodium chloride solution is preferably 10-27%, and further preferably 26.5%. Drying is preferably carried out using anhydrous sodium sulfate. The vacuum degree during rotary evaporation is preferably −0.05 to −0.01 MPa, and further preferably −0.03 MPa. The temperature for rotary evaporation is preferably 35-45° C., and further preferably 40° C. The rotary evaporation time is preferably 20-40 min, and further preferably 30 min. The eluent used for silica gel column chromatography is preferably n-hexane and dichloromethane, and the volume ratio of n-hexane to dichloromethane is preferably 92-98:5, and further preferably 95:5.

In the present application, the structural formula of compound E described in step (4) is as follows:

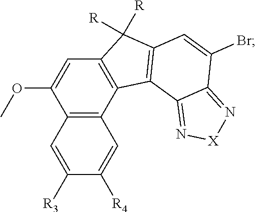

wherein R₃ and R₄ are each independently preferably selected from hydrogen, halogen, hydroxyl, alkyl, heterocycloalkyl, alkoxy, aryl, or heteroaryl; R is preferably selected from hydrogen, alkyl, cycloalkyl, or aryl; X is preferably N—R₉, O, or S; and R₉ is preferably selected from hydrogen, alkyl, aryl, or heteroaryl.

In the present application, the structural formula of compound F described in step (5) is as follows:

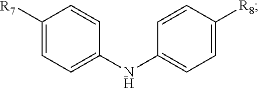

wherein R₇ and R₅ are each independently preferably selected from hydrogen, halogen, hydroxyl, alkyl, alkoxy, amino, aryl, or heteroaryl.

In step (5), the amounts of compound E, compound F, tris(dibenzylideneacetone) dipalladium (0), cesium carbonate, toluene, and water are preferably 15-20 mmol, 25-30 mmol, 0.5-1.5 mmol, 34-38 mmol, 250 g, and 20-30 g, respectively; further preferably 16-18.2 mmol, 27.2-29 mmol, 0.7-1.2 mmol, 35-37 mmol, 250 g, and 22-28 g, respectively; and more preferably 17 mmol, 28 mmol, 0.9 mmol, 36.5 mmol, 250 g, and 25 g, respectively. The temperature of the cross-coupling reaction is preferably 90-110° C., further preferably 95-105° C., and more preferably 100° C. The reaction time of the cross-coupling reaction is preferably 16-24 h, further preferably 18-22 h, and more preferably 20 h.

In the present application, the cross-coupling reaction described in step (5) is preferable carried out under a nitrogen atmosphere, and stirring is preferably performed during the reaction. The stirring speed is preferably 200-300 rpm, and further preferably 250 rpm.

In the present application, after completion of the cross-coupling reaction described in step (5), a mixed solution is obtained. Preferably, the mixed solution is poured into water, followed by sequential dilution, washing, drying, rotary evaporation for concentration, and silica gel column chromatography. The volume/molar ratio of water to compound E is preferably 200-1000 mL: 15-20 mmol, further preferably 400-600 mL: 16-18.2 mmol, and more preferably 500 mL: 17 mmol. Dilution is preferably carried out using dichloromethane, and the volume/molar ratio of dichloromethane to compound E is preferably 30-200 mL: 15-20 mmol, further preferably 50-100 mL: 16-18.2 mmol, and more preferably 80 mL: 17 mmol. Washing is preferably performed on the organic phase obtained after dilution, and the washing agents are preferably used sequentially as water and a sodium chloride solution, wherein the mass concentration of the sodium chloride solution is preferably 10-27%, and further preferably 26.5%. Drying is preferably carried out using anhydrous sodium sulfate. The vacuum degree during rotary evaporation is preferably −0.05 to −0.01 MPa, and further preferably −0.03 to −0.02 MPa. The temperature for rotary evaporation is preferably 30-60° C., and further preferably 40° C. The rotary evaporation time is preferably 20-40 min, and further preferably 30 min. The eluent used for silica gel column chromatography is preferably n-hexane and dichloromethane, and the volume ratio of n-hexane to dichloromethane is preferably 92-98:5, and further preferably 95:5.

In the present application, the structural formula of compound G described in step (5) is as follows:

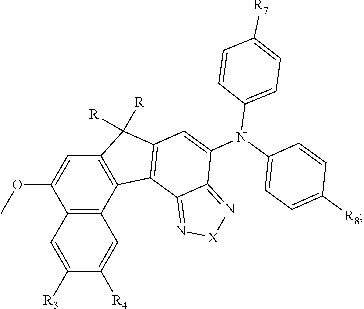

wherein R₃ and R₄ are each independently preferably selected from hydrogen, halogen, hydroxyl, alkyl, heterocycloalkyl, alkoxy, aryl, or heteroaryl; R₇ and R₅ are each independently preferably selected from hydrogen, halogen, hydroxyl, alkyl, alkoxy, amino, aryl, or heteroaryl; R is preferably selected from hydrogen, alkyl, cycloalkyl, or aryl; X is preferably N—R₉, O, or S; and R₉ is preferably selected from hydrogen, alkyl, aryl, or heteroaryl.

In step (6), the molar/volume ratio of compound G, boron tribromide, and chloroform is preferably 10-15 mmol: 15-20 mmol: 10-25 mL, further preferably 11-14 mmol: 16-19 mmol: 13-20 mL, and more preferably 12-13 mmol: 18 mmol: 18 mL. The temperature of the demethylation reaction is preferably 20-30° C., further preferably 22-28° C., and more preferably 25° C. The reaction time of the demethylation reaction is preferably 16-24 h, further preferably 18-22 h, and more preferably 20 h.

In the present application, during the demethylation reaction described in step (6), stirring is preferably performed. The stirring speed is preferably 150-250 rpm, and further preferably 200 rpm.

In the present application, after completion of the demethylation reaction described in step (6), a mixed solution is obtained. Preferably, the mixed solution is poured into a saturated sodium bicarbonate solution, followed by sequential dilution, extraction, washing, drying, rotary evaporation for concentration, and silica gel column chromatography. The volume/molar ratio of the saturated sodium bicarbonate solution to compound G is preferably 20-100 mL: 10-15 mmol, further preferably 40-60 mL: 11-14 mmol, and more preferably 50 mL: 12 mmol. Dilution is preferably carried out using dichloromethane, and the volume/molar ratio of dichloromethane to compound G is preferably 20-100 mL: 10-15 mmol, further preferably 40-60 mL: 11-14 mmol, and more preferably 50 mL: 12 mmol. During dilution, stirring is preferably performed, wherein the stirring speed is preferably 100-400 rpm, and further preferably 200 rpm, and the stirring time is preferably 5-15 min, and further preferably 10 min. Extraction is preferably performed on the aqueous phase obtained after dilution, and the extraction solvent is preferably dichloromethane. The number of extractions is preferably 2-4 times, and further preferably 3 times. For each extraction, the volume/molar ratio of the extraction solvent to compound G is preferably 20-100 mL: 10-15 mmol, further preferably 40-60 mL: 11-14 mmol, and more preferably 50 mL: 12 mmol. Washing is preferably performed on the organic phase obtained after extraction, and the washing agents are preferably used sequentially as water and a sodium chloride solution, wherein the mass concentration of the sodium chloride solution is preferably 10-27%, and further preferably 26.5%. Drying is preferably carried out using anhydrous sodium sulfate. The vacuum degree during rotary evaporation is preferably −0.05 to −0.01 MPa, and further preferably −0.03 MPa. The temperature for rotary evaporation is preferably 20-30° C., and further preferably 25° C. The rotary evaporation time is preferably 20-40 min, and further preferably 30 min. The eluent used for silica gel column chromatography is preferably n-hexane and dichloromethane, and the volume ratio of n-hexane to dichloromethane is preferably 4-6:5, and further preferably 5:5.

In the present application, the structural formula of compound H described in step (6) is as follows:

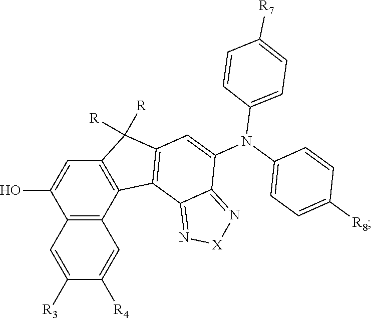

wherein R₃ and R₄ are each independently preferably selected from hydrogen, halogen, hydroxyl, alkyl, heterocycloalkyl, alkoxy, aryl, or heteroaryl; R₇ and R₅ are each independently preferably selected from hydrogen, halogen, hydroxyl, alkyl, alkoxy, amino, aryl, or heteroaryl; R is preferably selected from hydrogen, alkyl, cycloalkyl, or aryl; X is preferably N—R₉, O, or S; and R₉ is preferably selected from hydrogen, alkyl, aryl, or heteroaryl.

In the present application, the structural formula of compound J described in step (7) is as follows:

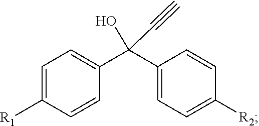

wherein R₁ and R₂ are each independently preferably selected from hydrogen, halogen, hydroxyl, alkyl, alkoxy, amino, aryl, or heteroaryl.

In step (7), the molar/volume ratio of compound H, compound J, pyridinium p-toluenesulfonate, and dichloromethane is preferably 2-5 mmol: 3-6 mmol: 1-4 mmol: 100 mL, further preferably 3-4 mmol: 4-5 mmol: 2-3 mmol: 100 mL, and more preferably 3.8 mmol: 4.7 mmol: 2.8 mmol: 100 mL. The reaction temperature is preferably 35-45° C., further preferably 38-42° C., and more preferably 40° C. The reaction time is preferably 4-10 h, further preferably 6-8 h, and more preferably 7 h.

In the present application, the reaction described in step (7) is preferably carried out under a nitrogen atmosphere, and stirring is preferably performed during the reaction. The stirring speed is preferably 100-200 rpm, and further preferably 150 rpm.

In the present application, after completion of the reaction described in step (7), a mixed solution is obtained. Preferably, the mixed solution is poured into ice water, followed by extraction to obtain an organic phase, and the organic phase is then sequentially subjected to rotary evaporation for concentration and silica gel column chromatography. The volume/molar ratio of ice water to compound His preferably 50-250 mL: 2-5 mmol, further preferably 100-200 mL: 3-4 mmol, and more preferably 150 mL: 3.8 mmol. The extraction solvent is preferably toluene, and the number of extractions is preferably 2-4 times, and further preferably 3 times. For each extraction, the volume/molar ratio of the extraction solvent to compound H is preferably 150-250 mL: 2-5 mmol, further preferably 170-220 mL: 3-4 mmol, and more preferably 200 mL: 3.8 mmol. The vacuum degree during rotary evaporation is preferably −0.05 to −0.01 MPa, and further preferably −0.03 to −0.02 MPa. The temperature for rotary evaporation is preferably 30-60° C., and further preferably 40° C. The rotary evaporation time is preferably 20-40 min, and further preferably 30 min. The eluent used for silica gel column chromatography is preferably petroleum ether and dichloromethane, and the volume ratio of petroleum ether to dichloromethane is preferably 0.5-2:1, and further preferably 1:1.

The present application further provides an application of the photochromic compound in photochromic products.

In the present application, the application of the photochromic compound in photochromic products preferably comprises preparing the photochromic compound into a photochromic composition, and then preparing the photochromic composition into a photochromic product.

In the present application, the photochromic composition preferably comprises the photochromic compound and an organic polymer. The organic polymer preferably comprises one or more selected from polystyrene, poly(methyl methacrylate), polycarbonate, polyurethane, epoxy resin, polyethylene, polyvinyl alcohol, polysiloxane, polyolefin, polyamide, polyester, polyacrylate, copolymers containing vinyl groups and/or vinyl acetate, cellulose acetate, cellulose acetate butyrate, and polyvinyl butyral, as well as homopolymers and/or copolymers derived from the above compounds.

In the present application, the photochromic composition preferably further comprises one or more selected from surfactants, photoinitiators, thermal initiators, polymerization inhibitors, solvents, light stabilizers, thermal stabilizers, mold release agents, defoamers, rheology modifiers, leveling agents, free radical scavengers, and adhesion promoters.

In the present application, the photochromic product preferably comprises lenses, optical lens products, glass products, or electronic consumer products. The optical lens products preferably comprise magnifying lenses, camera lenses, or light-adjusting lenses. The glass products preferably comprise photochromic interlayers, photochromic glass, or photochromic automotive films. The electronic consumer products preferably comprise display screens, display backplates, security elements, anti-counterfeiting elements, decorative products, clothing, or accessories.

The technical solutions provided by the present application are described in detail below in combination with the embodiments; however, these embodiments shall not be construed as limiting the scope of protection of the present application.

### Embodiment 1

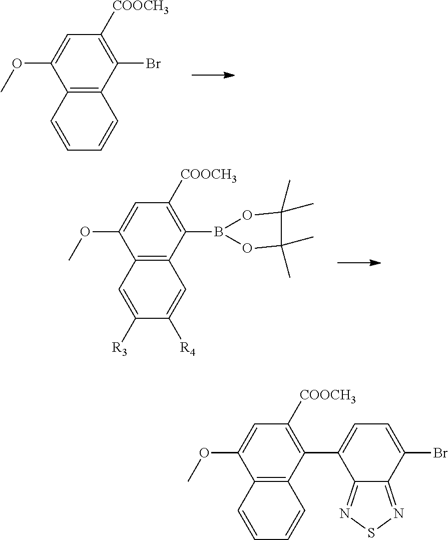

A mixture of 50.8 mmol of methyl 1-bromo-4-methoxy-2-naphthoate, 1 mmol of [1,1′-bis(diphenylphosphino)ferrocene]palladium (II) dichloride, 61 mmol of bis(pinacolato)diboron, 150 g of toluene, 13.5 g of water, and 1.5 g of potassium acetate was prepared. After purging with nitrogen, the temperature was raised to 80° C., and the reaction mixture was stirred at 200 rpm for 24 h to obtain a mixed solution. The mixed solution was cooled to room temperature and concentrated by rotary evaporation at −0.01 MPa and 50° C. for 30 min, thereby obtaining an intermediate product. The intermediate product (76.2 mmol), 14,7-dibromo-2,1,3-benzothiadiazole (2 mmol), tetrakis(triphenylphosphine)palladium (0) (2 mmol), 50 g of an aqueous potassium carbonate solution (with a concentration of 2 mol/L), and 250 g of tetrahydrofuran were mixed. After purging with nitrogen, the temperature was raised to 60° C., and the mixture was stirred at 200 rpm for 18 h to obtain a mixed solution. The mixed solution was poured into 200 mL of hydrochloric acid (with a concentration of 2 mol/L), followed by dilution with 500 mL of ethanol, to obtain an organic phase. The organic phase was sequentially washed with water and a sodium chloride solution having a mass concentration of 26.5%, dried over anhydrous sodium sulfate, and then concentrated by rotary evaporation at −0.01 MPa and 40° C. for 20 min. The resulting product was purified by silica gel column chromatography (silica gel particle size: 200-300 mesh) using n-hexane/dichloromethane (v/v=7:3) as the eluent, to obtain compound C, namely methyl 1-(7-(benzothiadiazolyl))-4-methoxy-2-naphthoate. Compound C was obtained as a yellow crystalline solid with a mass of 16.3 g, a two-step yield of 75%, and the following mass spectrometry data: MS (ESI, m/z): [M+H]⁺ calcd for C₁₉H₁₃BrN₂O₃ S, 429.0; found, 429.0.

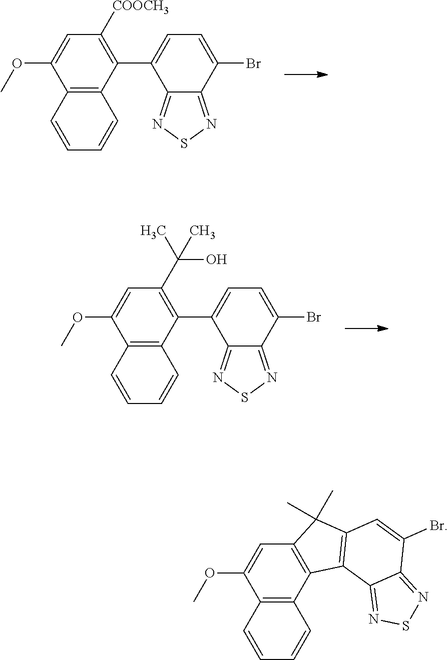 [SMILES: COC(=O)c1cc(OC)c2ccccc2c1-c1ccc(Br)c2nsnc12 ; COc1cc(C(C)(C)O)c(-c2ccc(Br)c3nsnc23)c2ccccc12 ; COc1cc2c(c3ccccc13)-c1c(cc(Br)c3nsnc13)C2(C)C]

34.9 mmol of methyl 1-(7-(benzothiadiazolyl))-4-methoxy-2-naphthoate and 250 mL of super-dry tetrahydrofuran were charged into a three-necked flask. After purging with nitrogen, 75 mL of an ethanolic solution of methylmagnesium bromide (the concentration of methylmagnesium bromide in the ethanolic solution being 1 mol/L) was added dropwise into the three-necked flask. The dropwise addition was completed within 30 min. The reaction mixture was stirred at 25° C. at a stirring speed of 200 rpm for 19 h, thereby obtaining a mixed solution. The mixed solution was poured into 200 mL of hydrochloric acid having a concentration of 2 mol/L, and 500 mL of ethanol was added for dilution. The mixture was stirred at 200 rpm for 10 min to obtain an organic phase. The organic phase was washed sequentially with water and a sodium chloride solution having a mass concentration of 26.5%, and then dried over anhydrous sodium sulfate. Thereafter, the solvent was removed by rotary evaporation at −0.01 MPa and 40° C. for 20 min. The residue was purified by silica gel column chromatography (silica gel particle size: 200-300 mesh) using n-hexane/dichloromethane (v/v=6:4) as the eluent, to obtain compound D. Compound D, 100 mL of chloroform, and 14 mmol of pyridinium p-toluenesulfonate were mixed, and the mixture was stirred at 25° C. at 200 rpm for 2 h to obtain a mixed solution. The mixed solution was poured into 500 mL of water, and the mixture was stirred at 200 rpm for 10 min to obtain an organic phase. The organic phase was washed with a sodium chloride solution having a mass concentration of 26.5%, dried over anhydrous sodium sulfate, and then concentrated by rotary evaporation at −0.01 MPa and 40° C. for 30 min. The residue was purified by silica gel column chromatography (silica gel particle size: 200-300 mesh) using n-hexane/dichloromethane (v/v=95:5) as the eluent, to obtain compound E: 4-bromo-8-methoxy-6,6-dimethyl-6H-indeno[5,6]thiadiazol. Compound E was obtained as a light orange solid with a mass of 7.6 g and a two-step yield of 53%. Mass spectrometry data: MS (ESI, m/z): [M+H]⁺ calcd for (C₂₀H₁₅BrN₂OS), 411.0; found, 411.0.

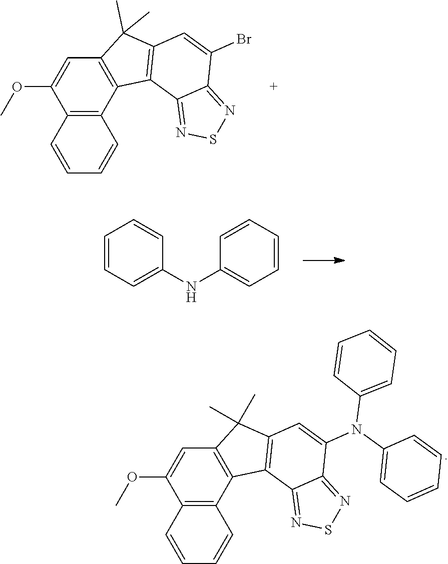 [SMILES: COc1cc2c(c3ccccc13)-c1c(cc(Br)c3nsnc13)C2(C)C ; c1ccc(Nc2ccccc2)cc1 ; COc1cc2c(c3ccccc13)-c1c(cc(N(c3ccccc3)c3ccccc3)c3nsnc13)C2(C)C]

A mixture of 18.2 mmol of 4-bromo-8-methoxy-6,6-dimethyl-6H-indeno[5,6]thiadiazole, 0.9 mmol of tris(dibenzylideneacetone)dipalladium (0), 36.5 mmol of cesium carbonate, 27.2 mmol of diphenylamine, 250 g of toluene, and 25 g of water was prepared. After purging with nitrogen, the temperature was raised to 100° C., and the reaction mixture was stirred at a stirring speed of 250 rpm for 20 h to obtain a mixed solution. The mixed solution was cooled to room temperature and then poured into 500 mL of water, followed by addition of 80 mL of dichloromethane. The mixture was stirred at 250 rpm for 10 min to obtain an organic phase. The organic phase was washed sequentially with water and a sodium chloride solution having a mass concentration of 26.5%, dried over anhydrous sodium sulfate, and then concentrated by rotary evaporation at −0.01 MPa and 40° C. for 20 min. The residue was purified by silica gel column chromatography (silica gel particle size: 200-300 mesh) using n-hexane/dichloromethane (v/v=95:5) as the eluent, to obtain compound G, namely 8-methoxy-6,6-dimethyl-4-diphenylamino-6H-indeno[5,6]thiadiazole. Compound G was obtained as an off-white solid with a mass of 6.1 g, a yield of 67%, and the following mass spectrometry data: MS (ESI, m/z): [M+H]⁺ calcd for C₃₂H₂₅N₃OS, 500.6; found, 500.4.

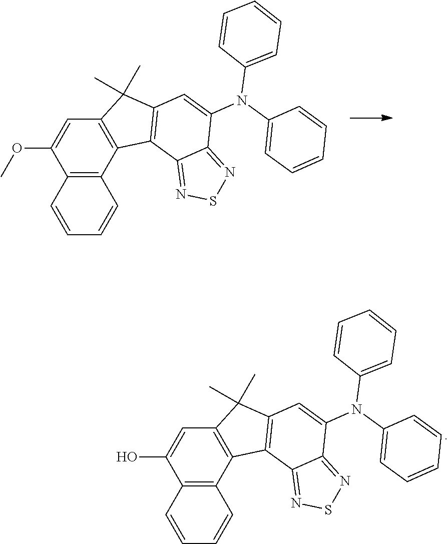 [SMILES: COc1cc2c(c3ccccc13)-c1c(cc(N(c3ccccc3)c3ccccc3)c3nsnc13)C2(C)C ; CC1(C)c2cc(O)c3ccccc3c2-c2c1cc(N(c1ccccc1)c1ccccc1)c1nsnc21]

A mixture of 12 mmol of 8-methoxy-6,6-dimethyl-4-diphenylamino-6H-indeno[5,6]thiadiazole and 18 mL of chloroform was prepared. 18 mL of a dichloromethane solution of boron tribromide (the concentration of boron tribromide in the dichloromethane solution being 1 mol/L) was added dropwise at a rate of 0.6 mL/min. After completion of the dropwise addition, the reaction mixture was stirred at 25° C. and 200 rpm for 20 h, thereby obtaining a mixed solution. The mixed solution was poured into 50 mL of a saturated sodium bicarbonate solution, followed by addition of 50 mL of dichloromethane, and the mixture was stirred at 200 rpm for 10 min to obtain an aqueous phase. The aqueous phase was extracted with 50 mL of dichloromethane each time, and the extraction was performed three times to obtain an organic phase. The organic phase was sequentially washed with water and a sodium chloride solution having a mass concentration of 26.5%, dried over anhydrous sodium sulfate, and then concentrated by rotary evaporation at −0.01 MPa and 25° C. for 30 min. The residue was purified by silica gel column chromatography (silica gel particle size: 200-300 mesh) using n-hexane/dichloromethane (v/v=5:5) as the eluent, to obtain compound H, namely 8-hydroxy-6,6-dimethyl-4-diphenylamino-6H-indeno[5,6]thiadiazole. Compound H was obtained as a gray solid with a mass of 5 g, a yield of 86%, and the following mass spectrometry data: MS (ESI, m/z): [M+H]⁺ calcd for C₃₁H₂₃N₃OS, 486.2; found, 486.1.

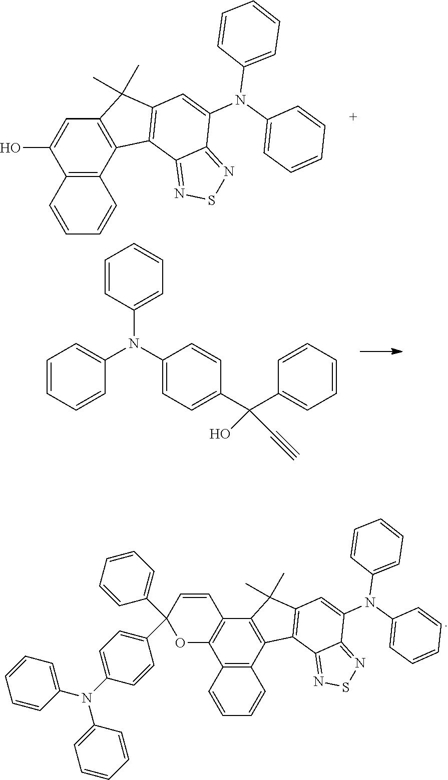

A mixture of 4.7 mmol of 1-(4-diphenylaminophenyl)-1-phenyl-2-propyn-1-one, 3.8 mmol of 8-hydroxy-6,6-dimethyl-4-diphenylamino-6H-indeno[5,6]thiadiazole, and 100 mL of dichloromethane was prepared, followed by addition of 2.8 mmol of pyridinium p-toluenesulfonate. After purging with nitrogen, the temperature was raised to 40° C., and the reaction mixture was stirred at a stirring speed of 150 rpm for 6 h, thereby obtaining a mixed solution. The mixed solution was poured into 200 mL of ice water, and extracted with 200 mL of toluene each time. The extraction was performed three times to obtain an organic phase. The organic phase was concentrated by rotary evaporation at −0.01 MPa and 40° C. for 20 min. The residue was purified by silica gel column chromatography (silica gel particle size: 200-300 mesh) using petroleum ether/dichloromethane (v/v=1:1) as the eluent, to obtain the photochromic compound, namely 3-(4-diphenylaminophenyl)-3-phenyl-13,13-dimethyl-11-diphenylamino-2-azafluorene [8,9]thiadiazole. The photochromic compound was obtained with a mass of 960 mg, a yield of 29%, and a purity of 99.5%. Mass spectrometry data: MS (ESI, m/z): [M+H]⁺ calcd for C₅₈H₄₂N₄OS, 844.1; found, 844.1. ¹H NMR data: 1H NMR (400 MHz, CDCl₃) δ 8.71 (d, 1H), 8.49 (d, 1H), 8.17 (d, 1H), 7.63 (d, 1H), 7.59-7.49 (m, 5H), 7.43-7.38 (m, 4H), 7.34-7.30 (m, 2H), 7.29-7.24 (m, 5H), 7.24-7.18 (m, 6H), 7.14-7.06 (m, 8H), 6.89 (d, 1H), 6.28 (d, 1H), 2.10 (s, 6H).

### Embodiment 2

In Example 1, methyl 1-bromo-4-methoxy-2-naphthoate was replaced with methyl 1-bromo-4,6,7-trimethoxy-2-naphthoate, diphenylamine was replaced with 4,4 ‘-dimethoxydiphenylamine, and 1-(4-diphenylaminophenyl)-1-phenyl-2-propyn-1-one was replaced with 1-(4-(4-methoxyphenyl)diphenylaminophenyl)-1-phenyl-2-propyn-1-one, while all other conditions were the same as those in Example 1.

Mass spectrometry data of the photochromic compound prepared in this example: MS (ESI, m/z): [M+H]⁺ calcd for C₆₄H₅₄N₄O₇ S, 1023.4; found, 1023.4.

The structural formula of the photochromic compound prepared in this example is as follows:

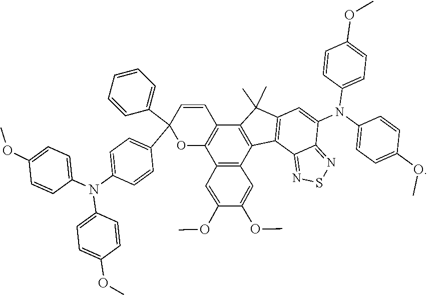 [SMILES: COc1ccc(N(c2ccc(OC)cc2)c2ccc(C3(c4ccccc4)C=Cc4c5c(c6cc(OC)c(OC)cc6c4O3)-c3c(cc(N(c4ccc(OC)cc4)c4ccc(OC)cc4)c4nsnc34)C5(C)C)cc2)cc1]

### Embodiment 3

In Example 2, 4,4’-dimethoxydiphenylamine is replaced with 4,4′-bis(N,N-dimethylamino)diphenylamine, while all other conditions are the same as those in Example 2.

Mass spectrometry data of the photochromic compound prepared in this example: MS (ESI, m/z): [M+H]⁺ calcd for C₆₆H₆₀N₆O₅ S, 1050.3; found, 1050.3

The structural formula of the photochromic compound prepared in this example is as follows:

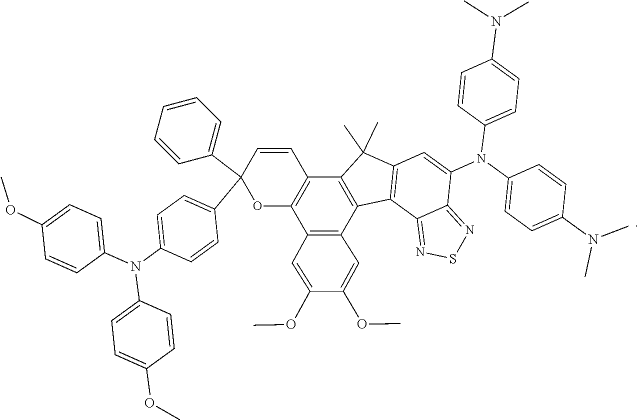

### Embodiment 4

In Example 1, methylmagnesium bromide is replaced with phenylmagnesium bromide, while all other conditions are the same as those in Example 1.

Mass spectrometry data of the photochromic compound prepared in this example: MS (ESI, m/z): [M+H]⁺ calcd for C₆₈H₄₆N₄OS, 967.3; found, 967.3.

The structural formula of the photochromic compound prepared in this example is as follows:

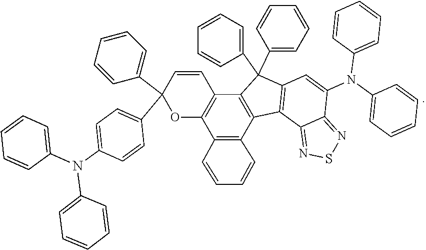 [SMILES: C1=CC(c2ccccc2)(c2ccc(N(c3ccccc3)c3ccccc3)cc2)Oc2c1c1c(c3ccccc23)-c2c(cc(N(c3ccccc3)c3ccccc3)c3nsnc23)C1(c1ccccc1)c1ccccc1]

### Embodiment 5

In Example 1, 4,7-dibromo-2,1,3-benzothiadiazole is replaced with 4,7-dibromo-2,1,3-benzoxadiazole, and methyl 1-bromo-4-methoxy-2-naphthoate is replaced with methyl 1-bromo-4,6,7-trimethoxy-2-naphthoate, while all other conditions are the same as those in Example 1.

Mass spectrometry data of the photochromic compound prepared in this example: MS (ESI, m/z): [M+H]⁺ calcd for C₆₂H₅₀N₄O₆, 947.4; found, 947.4.

The structural formula of the photochromic compound prepared in this example is as follows:

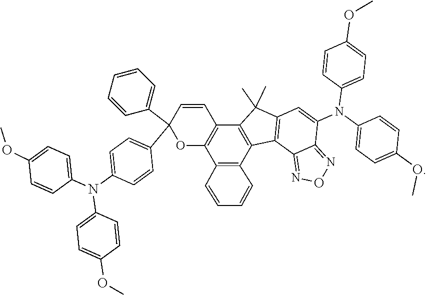 [SMILES: COc1ccc(N(c2ccc(OC)cc2)c2ccc(C3(c4ccccc4)C=Cc4c5c(c6ccccc6c4O3)-c3c(cc(N(c4ccc(OC)cc4)c4ccc(OC)cc4)c4nonc34)C5(C)C)cc2)cc1]

### Embodiment 6

In Example 1, 4,7-dibromo-2,1,3-benzothiadiazole is replaced with 4,7-dibromo-2-(n-butyl)-2H-benzo[d][1,2,3]triazole, diphenylamine is replaced with 4,4′-diethylaminodiphen ylamine, and 1-(4-diphenylaminophenyl)-1-phenyl-2-propyn-1-one is replaced with 1-(4-(4-di ethylaminophenyl)diphenylaminophenyl)-1-phenyl-2-propyn-1-one, while all other conditions are the same as those in Example 1.

Mass spectrometry data of the photochromic compound prepared in this example: MS (ESI, m/z): [M+H]⁺ calcd for C₈₈H₉₁N₆O, 1050.3; found, 1050.3.

The photochromic compound prepared in this example has the following structural formula:

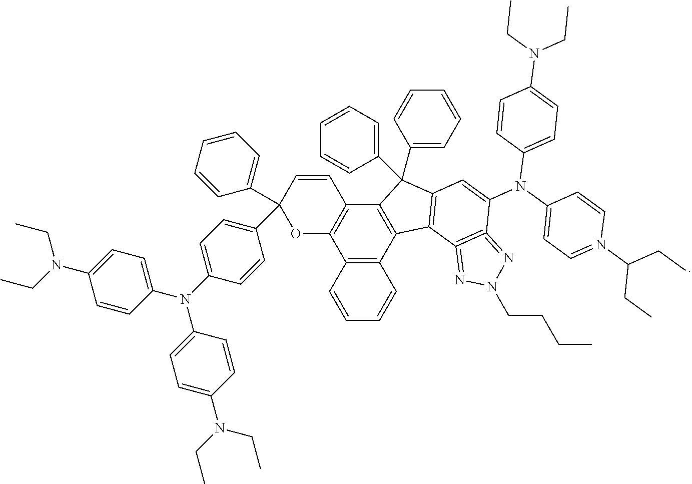

### Comparative Example 1

The structural formula of the photochromic compound is as follows:

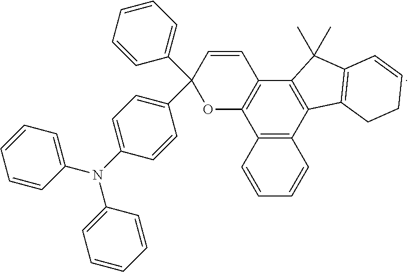 [SMILES: CC1(C)C2=C(CCC=C2)c2c1c1c(c3ccccc23)OC(c2ccccc2)(c2ccc(N(c3ccccc3)c3ccccc3)cc2)C=C1]

### Application Example 1

In terms of parts by mass, 25 parts of polyvinyl butyral (PVB) resin, 6 parts of diisobutyl phthalate, 65 parts of ethyl acetate, 2 parts of bis(2,2,6,6-tetramethyl-4-piperidyl) sebacate, 1 part of 9,10-dihydro-9-oxa-10-phosphaphenanthrene-10-oxide, 0.5 part of di-tert-butyl hydroxytoluene, and 0.5 part of the photochromic compound prepared in Example 1 are mixed. The mixture is mechanically stirred at 1000 rpm for 2 hours, followed by standing for 1 hour, to obtain a mixed material. The mixed material is placed into an extruder and extruded at 140° C., and then hot-pressed at 140° C. and 1.0 MPa to form a film having a thickness of 3.6 μm. After natural cooling to room temperature, a photochromic interlayer film is obtained.

Two pieces of transparent clear glass with dimensions of 10 cm×10 cm are taken. The photochromic interlayer film is placed between the two pieces of transparent clear glass, and pre-pressing is carried out using a rolling press device (pre-pressing temperature: 70° C.; pressure: 0.1 MPa) to preliminarily remove air bubbles. Thereafter, pressing is carried out at 130° C. and 0.8 MPa for 90 minutes to form the laminate. After natural cooling to room temperature, laminated double-layer glass with an interlayer is obtained.

### Comparative Application Example 1

The photochromic compound in Application Example 1 is replaced with the photochromic compound of Comparative Example 1, while all other conditions are the same as those in Application Example 1.

Irradiation testing of the laminated double-layer glass is carried out in accordance with GB/T 5137.3-2020. Near-infrared absorption testing of the laminated double-layer glass is carried out in accordance with ISO 9050:2017, with a wavelength range of 780-2500 nm. The test results are shown in Table 1.

TABLE 1
Results of irradiation and near-infrared absorption
tests of laminated double- layer glass.

| | Visible-Light Transmittance | | Near-Infrared Absorption | |
|---|---|---|---|---|
| | Before/After Color Change | | Before/After Color Change | |
| | Before | After | Before | After |
| | Irradiation | Irradiation | Irradiation | Irradiation |
| Application | 70% | 68% | Has infrared | Has infrared |
| Example 1 | | | Absorption | Absorption |
| Comparative | 30% | 15% | NA | NA |
| Application | | | | |
| Example | | | | |

The above descriptions are only preferred embodiments of the present application. It should be pointed out that, for a person of ordinary skill in the art, various improvements and modifications may be made without departing from the principles of the present application, and such improvements and modifications shall also fall within the scope of protection of the present application.

What is claimed is:

1. A photochromic compound, wherein the structural formula of the photochromic compound is as follows: 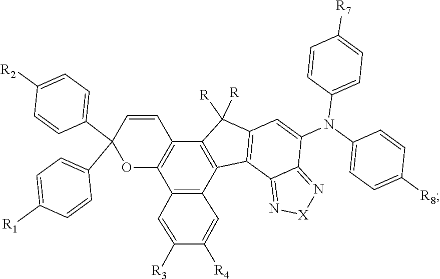
wherein the photochromic compound is any one of the compounds represented by the following structural formulas:

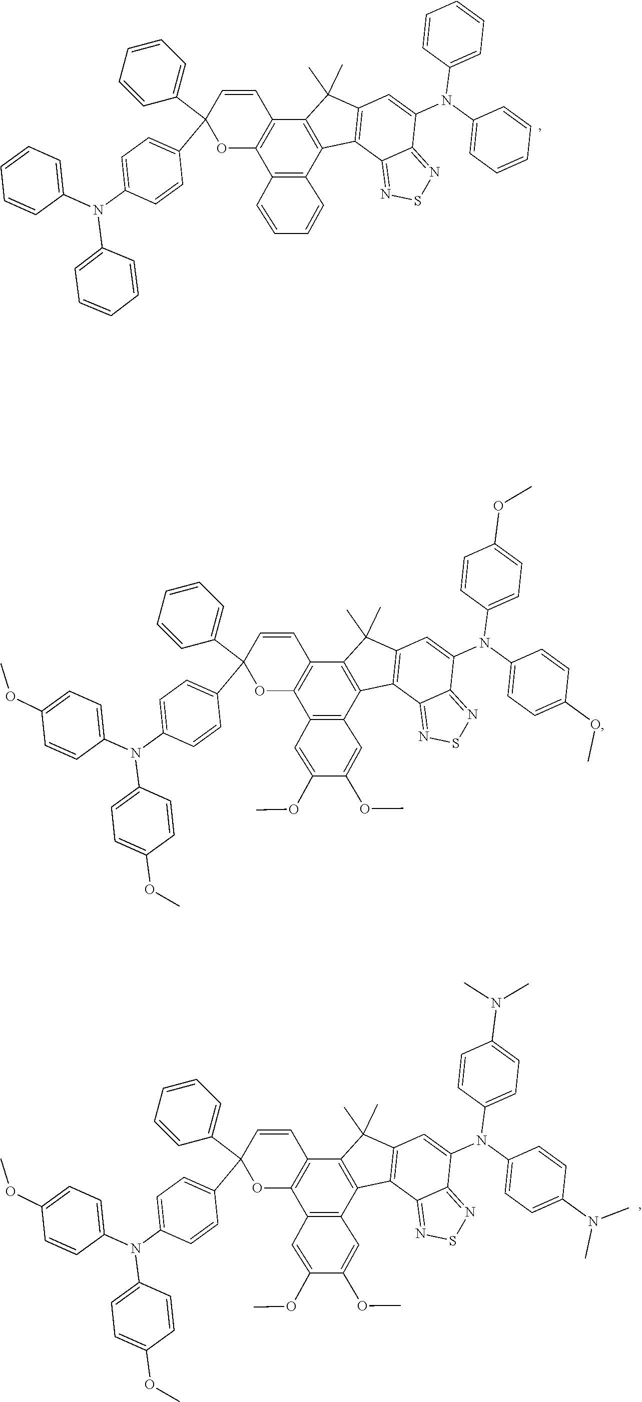

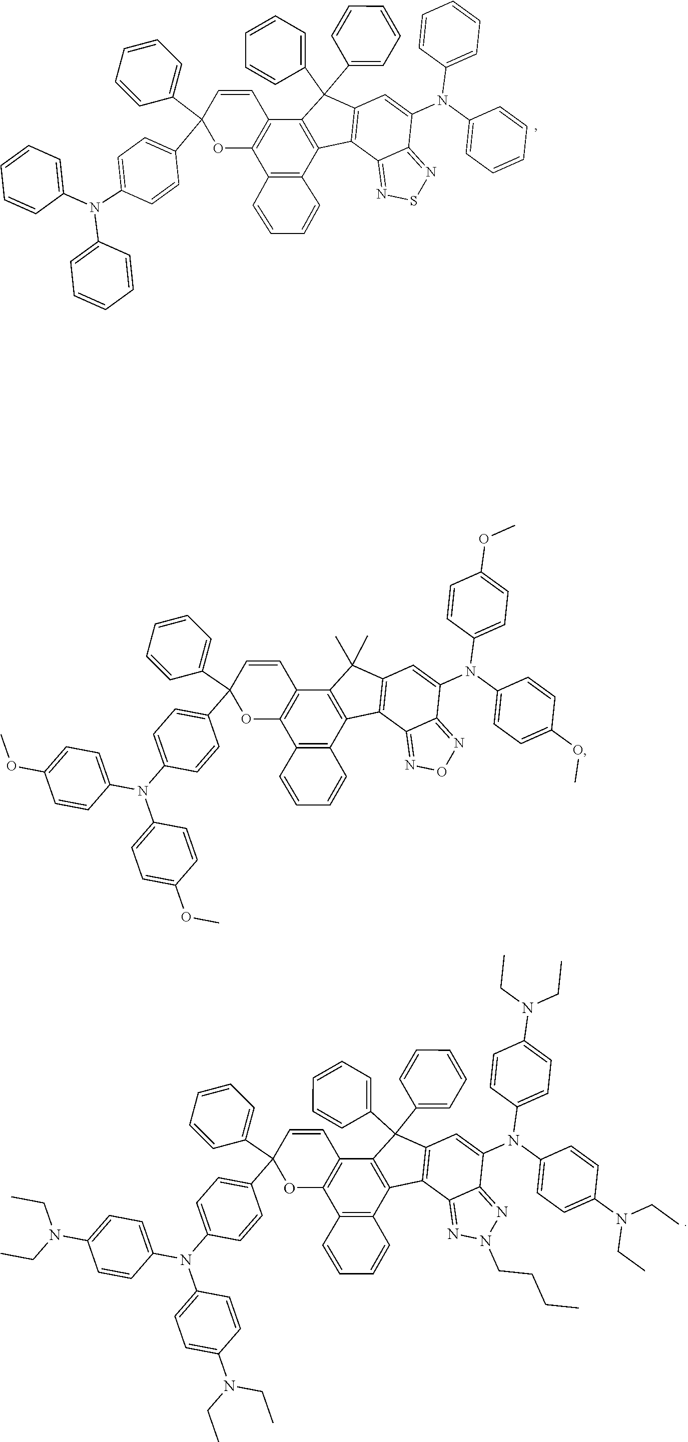 [SMILES: C1=CC(c2ccccc2)(c2ccc(N(c3ccccc3)c3ccccc3)cc2)Oc2c1c1c(c3ccccc23)-c2c(cc(N(c3ccccc3)c3ccccc3)c3nsnc23)C1(c1ccccc1)c1ccccc1 ; COc1ccc(N(c2ccc(OC)cc2)c2ccc(C3(c4ccccc4)C=Cc4c5c(c6ccccc6c4O3)-c3c(cc(N(c4ccc(OC)cc4)c4ccc(OC)cc4)c4nonc34)C5(C)C)cc2)cc1 ; CCCCn1nc2c(N(c3ccc(N(CC)CC)cc3)c3ccc(N(CC)CC)cc3)cc3c(c2n1)-c1c(c2c(c4ccccc14)OC(c1ccccc1)(c1ccc(N(c4ccc(N(CC)CC)cc4)c4ccc(N(CC)CC)cc4)cc1)C=C2)C3(c1ccccc1)c1ccccc1]

2. A method for preparing the photochromic compound, comprises the following steps:
(1) compound A, [1,1,-bis(diphenylphosphino)ferrocene]palladium (II) dichloride (Pd(dppf)Cl₂), bis(pinacolato)diboron, potassium acetate, toluene, and water are subjected to a Miyaura borylation reaction to afford an intermediate product;
(2) the intermediate product, compound B, tetrakis(triphenylphosphine) palladium (0) (Pd(PPh3)₄), an aqueous potassium carbonate solution, and tetrahydrofuran are subjected to a Suzuki coupling reaction to afford compound C;
(3) compound C, a Grignard reagent, and tetrahydrofuran are mixed and allowed to react to afford compound D;
(4) compound D, pyridinium p-toluenesulfonate (PPTS), and chloroform are subjected to a cyclization reaction to afford compound E;
(5) compound E, compound F, tris(dibenzylideneacetone)dipalladium (0) (Pd₂(dba)₃), cesium carbonate, toluene, and water are subjected to a cross-coupling reaction to afford compound G;
(6) compound G, boron tribromide (BBr₃), and chloroform are subjected to a demethylation reaction to afford compound H;
(7) compound H, compound J, pyridinium p-toluenesulfonate (PPTS), and dichloromethane are mixed and allowed to react, thereby obtaining the photochromic compound;
wherein structural formula of compound A described in step (1) is as follows: 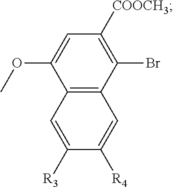
wherein the structural formula of the intermediate product described in step (1) is as follows: 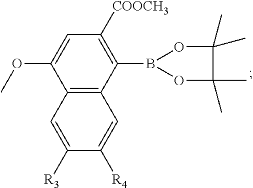
wherein the structural formula of compound B described in step (2) is as follows: 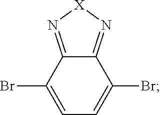
wherein the structural formula of compound C described in step (2) is as follows: 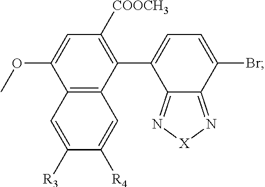
wherein the structural formula of compound D described in step (3) is as follows: 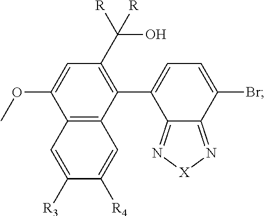
wherein the structural formula of compound E described in step (4) is as follows: 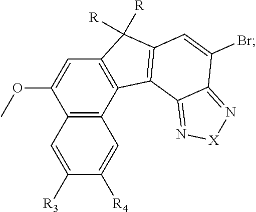
wherein the structural formula of compound F described in step (5) is as follows: 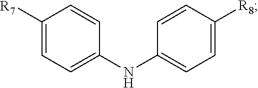
wherein the structural formula of compound G described in step (5) is as follows: 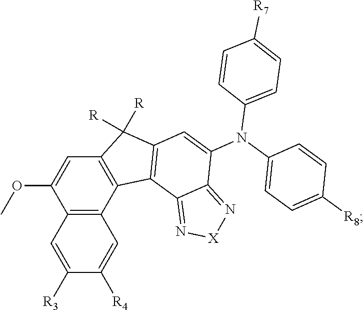
wherein the structural formula of compound H described in step (6) is: 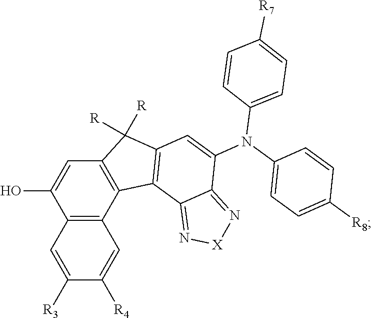
wherein the structural formula of compound J described in step (7) is:

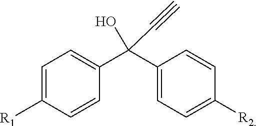

3. The method according to claim 2, wherein the molar ratio of compound A, [1,1′-bis(diphenylphosphino)ferrocene]dichloropalladium, and bis(pinacolato)diboron described in step (1) is 48-52:1:58-63; wherein the mass-to-mole ratio of compound A, potassium acetate, toluene, and water is 48-52 mmol: 1-2 g: 120-180 g: 10-15 g; wherein the temperature of the Miyaura borylation reaction is 70-90° C., and the time of the Miyaura borylation reaction is 20-28 h.

4. The method according to claim 2, wherein the molar ratio of compound B in step (2), tetrakis(triphenylphosphine)palladium, and compound A described in step (1) is 75-80:2:48-52; wherein the mass-to-mole ratio of the potassium carbonate solution, tetrahydrofuran, and compound A described in step (1) is 48-52 g: 200-300 g: 48-52 mmol; wherein the concentration of the potassium carbonate solution is 1.5-2.5 mol/L; wherein the temperature of the Suzuki coupling reaction is 50-70° C., and the time of the Suzuki coupling reaction is 12-24 h.

5. The method according to claim 3, wherein the molar ratio of compound B in step (2), tetrakis(triphenylphosphine)palladium, and compound A described in step (1) is 75-80:2:48-52; wherein the mass-to-mole ratio of the potassium carbonate solution, tetrahydrofuran, and compound A described in step (1) is 48-52 g: 200-300 g: 48-52 mmol; wherein the concentration of the potassium carbonate solution is 1.5-2.5 mol/L; wherein the temperature of the Suzuki coupling reaction is 50-70° C., and the time of the Suzuki coupling reaction is 12-24 h.

6. The method according to claim 4, wherein the structural formula of the Grignard reagent described in step (3) is R—Mg—Br;
wherein the molar-to-volume ratio of compound C, Grignard reagent, and tetrahydrofuran described in step (3) is 32-38 mmol: 72-78 mmol: 250 mL; wherein the reaction temperature is 20-30° C., and the reaction time is 14-24 h;
wherein the molar ratio of pyridinium p-toluenesulfonate described in step (4) to compound C described in step (3) is 12-16:32-38; wherein the molar-to-volume ratio of pyridinium p-toluenesulfonate to chloroform is 12-16 mmol: 100 mL; wherein the temperature of the cyclization reaction is 20-30° C., and the time of the cyclization reaction is 1-3 h.

7. The method according to claim 5, wherein the structural formula of the Grignard reagent described in step (3) is R—Mg—Br;
wherein the molar-to-volume ratio of compound C, Grignard reagent, and tetrahydrofuran described in step (3) is 32-38 mmol: 72-78 mmol: 250 mL; wherein the reaction temperature is 20-30° C., and the reaction time is 14-24 h;
wherein the molar ratio of pyridinium p-toluenesulfonate described in step (4) to compound C described in step (3) is 12-16:32-38; wherein the molar-to-volume ratio of pyridinium p-toluenesulfonate to chloroform is 12-16 mmol: 100 mL; wherein the temperature of the cyclization reaction is 20-30° C., and the time of the cyclization reaction is 1-3 h.

8. The method according to claim 7, wherein R₇ and R₈ of the structural formula of the photochromic compound of the present application are each independently selected from hydrogen, halogen, hydroxyl, alkyl, alkoxy, amino, aryl, or heteroaryl;
wherein the molar-to-mass ratio of compound E, compound F, tris(dibenzylideneacetone)dipalladium, cesium carbonate, toluene, and water described in step (5) is 15-20 mmol: 25-30 mmol: 0.5-1.5 mmol: 34-38 mmol: 250 g: 20-30 g;
wherein the temperature of the cross-coupling reaction is 90-110° C., and the time of the cross-coupling reaction is 16-24 h.

9. The method according to claim 8, wherein R₇ and R₈ of the structural formula of the photochromic compound of the present application are each independently selected from hydrogen, halogen, hydroxyl, alkyl, alkoxy, amino, aryl, or heteroaryl;
wherein the molar-to-mass ratio of compound E, compound F, tris(dibenzylideneacetone)dipalladium, cesium carbonate, toluene, and water described in step (5) is 15-20 mmol: 25-30 mmol: 0.5-1.5 mmol: 34-38 mmol: 250 g: 20-30 g; wherein the temperature of the cross-coupling reaction is 90-110° C., and the time of the cross-coupling reaction is 16-24 h.

10. The method according to claim 6, wherein the molar-to-volume ratio of compound G, boron tribromide, and chloroform described in step (6) is 10-15 mmol: 15-20 mmol: 10-25 mL; wherein the temperature of the demethylation reaction is 20-30° C., and the time of the demethylation reaction is 16-24 h.

11. The method according to claim 7, wherein the molar-to-volume ratio of compound G, boron tribromide, and chloroform described in step (6) is 10-15 mmol: 15-20 mmol: 10-25 mL; wherein the temperature of the demethylation reaction is 20-30° C., and the time of the demethylation reaction is 16-24 h.

12. The method according to claim 8, wherein the molar-to-volume ratio of compound G, boron tribromide, and chloroform described in step (6) is 10-15 mmol: 15-20 mmol: 10-25 mL; wherein the temperature of the demethylation reaction is 20-30° C., and the time of the demethylation reaction is 16-24 h.

13. The method according to claim 9, wherein the molar-to-volume ratio of compound G, boron tribromide, and chloroform described in step (6) is 10-15 mmol: 15-20 mmol: 10-25 mL; wherein the temperature of the demethylation reaction is 20-30° C., and the time of the demethylation reaction is 16-24 h.

14. The method according to claim 10, wherein the molar-to-volume ratio of compound H, compound J, pyridinium p-toluenesulfonate, and dichloromethane described in step (7) is 2-5 mmol: 3-6 mmol: 1-4 mmol: 100 mL; wherein the reaction temperature is 35-45° C., and the reaction time is 4-10 h.

15. The method according to claim 11, wherein the molar-to-volume ratio of compound H, compound J, pyridinium p-toluenesulfonate, and dichloromethane described in step (7) is 2-5 mmol: 3-6 mmol: 1-4 mmol: 100 mL; wherein the reaction temperature is 35-45° C., and the reaction time is 4-10 h.

16. The method according to claim 12, wherein the molar-to-volume ratio of compound H, compound J, pyridinium p-toluenesulfonate, and dichloromethane described in step (7) is 2-5 mmol: 3-6 mmol: 1-4 mmol: 100 mL; wherein the reaction temperature is 35-45° C., and the reaction time is 4-10 h.

17. The method according to claim 13, wherein the molar-to-volume ratio of compound H, compound J, pyridinium p-toluenesulfonate, and dichloromethane described in step (7) is 2-5 mmol: 3-6 mmol: 1-4 mmol: 100 mL; wherein the reaction temperature is 35-45° C., and the reaction time is 4-10 h.
